(19) United States
(12) Patent Application Publication
(10) Pub. No.: US 2026/0335580 A1
(43) Pub. Date: Oct. 29, 2026

(54) LARYNGEAL TUBE

(71) Applicant: VBM Medizintechnik GmbH, Sulz am Neckar (DE)

(72) Inventor: Volker Bertram, Sulz am Neckar (DE)

(73) Assignee: VBM Medizintechnik GmbH, Sulz am Neckar (DE)

(21) Appl. No.: 19/396,521

(22) Filed: Nov. 21, 2025

(30) Foreign Application Priority Data
Nov. 22, 2024 (DE) 10 2024 134 543.8

(51) Int. Cl. A61M 16/04

(52) U.S. Cl. CPC A61M 16/0486; A61M 16/0434; A61M 16/0493

(57) ABSTRACT

The invention relates to a laryngeal tube (10) and proposes an improved design which has a positive effect on the use and manufacture of the laryngeal tube (10).

### CROSS-REFERENCE TO RELATED APPLICATIONS

This application claims priority to DE Patent Application No. 10 2024 134 543.8 filed on Nov. 22, 2024, the entire contents of which are hereby incorporated by reference.

### DESCRIPTION

The invention relates to a laryngeal tube.

The laryngeal tube is a supraglottic airway (above the vocal cords (glottis) ) for temporary airway provision or management. In emergency situations, the laryngeal tube serves as an aid for airway management, as an alternative technique to mask ventilation and endotracheal intubation.

Typically, a laryngeal tube comprises a tube body having a ventilation lumen. An inflatable esophageal cuff, i.e., an inflatable blocking cuff, is arranged on the tube body for blocking the esophagus. The esophageal cuff is also referred to in the following as the distal cuff. A pharyngeal cuff is also arranged on the tube body for blocking the pharynx. The pharyngeal cuff is also referred to in the following as the proximal cuff. A ventilation opening of the ventilation lumen is arranged between the two cuffs.

For airway management, the laryngeal tube is inserted from the mouth into the pharynx (throat) until the ventilation opening of the tube body is in front of the laryngeal inlet of the person to be ventilated. Subsequently, the two cuffs are inflated such that the distal esophageal cuff seals the esophagus, and the proximally arranged pharyngeal cuff seals the oropharynx.

The laryngeal tubes sometimes have an additional drainage channel to relieve pressure on the stomach, i.e., to drain gastric contents. The drainage channel has a drainage opening arranged distally to the esophageal cuff. This can counteract the risk of aspiration (ingress of a liquid or solid substance or mixture into the trachea and lower respiratory tract) as well as the risk of injury (rupture) of the esophagus associated with vomiting.

Laryngeal tubes are known, for example, from DE 10 2011 001 325 B4 and U.S. Pat. No. 4,231,365.

WO 2011/106754 A1 describes a multi-piece laryngeal tube in which the tube body has a proximal shaft longitudinal portion and a distal shaft longitudinal portion which are interconnected. The ventilation lumen has a circular passage cross-section and a ventilation opening arranged laterally on the distal shaft longitudinal portion of the tube body. A further laryngeal tube is known from US 2005/0229933 A1.

The object of the present invention is to provide a laryngeal tube which has improved properties with regard to its use and preferably also offers improved manufacturability.

For this purpose, the invention proposes a laryngeal tube according to the following aspects, each of which has advantages over the known tubes. The features that characterize a laryngeal tube of one aspect are also to be understood as possible further developments of the laryngeal tube of another aspect.

The aspects of the present invention are based upon a laryngeal tube having the following common features: Laryngeal tube for ventilating a patient. The laryngeal tube is designed having a tube body which is designed having a ventilation channel, a drainage channel, and an aeration channel. An esophageal cuff is arranged on the tube body to block the esophagus. Further, a pharyngeal cuff is arranged on the tube body to block the pharynx. The drainage channel has a drainage opening located distally from the pharyngeal cuff, and the ventilation channel has a ventilation opening located between the pharyngeal cuff and the esophageal cuff. The esophageal cuff and/or the pharyngeal cuff can be inflated and/or deflated via the aeration channel.

The tube body is elongated and extends along a central longitudinal line. Typically, the tube body or the central longitudinal line thereof is curved at least in portions. The curvature of the tube body is designed, in particular, such that the center or centers of curvature lie on one side of the tube body. The tube body is therefore curved only in one direction.

The tube body is, in particular, flexible; in particular, it comprises (in particular, predominantly) a resilient material or is formed thereby. This makes the laryngeal tube easy to insert and minimizes the risk of injury when inserted into the patient's pharynx.

According to a first aspect, such a laryngeal tube is characterized in that the tube body has a tube shaft. The tube shaft refers to a subportion of the tube body without the tip thereof. In the tube shaft, the breathing channel is arranged centrally and the drainage channel is arranged on a first side of the ventilation channel, and the aeration channel is arranged on a second side of the ventilation channel that is opposite the first side, such that the tube shaft has a flattened cross-sectional shape. The channels are therefore virtually on one “plane.” The flattened cross-sectional shape results in a tubular shaft that has narrow sides and broad sides. In the tube shaft, the ventilation channel, the aeration channel, and the drainage channel run, in particular, parallel to one another. The tube shaft typically ends at the distal end of the ventilation opening, since the ventilation channel typically extends distally only to the ventilation opening or opens into the surface of the tube body via the ventilation opening.

When looking at a cross-section of the tube shaft along the central longitudinal line, which runs orthogonally to the cross-sectional area, the openings of the drainage channel, ventilation channel, and aeration channel can be seen to be located, in particular, on a straight line, or a straight line can be drawn through the openings of all three channels in the cross-sectional area. In particular, the areal center points of the openings of all three channels lie on a line in the cross-sectional area.

The ventilation channel has, in particular, the largest clear cross-sectional area, in particular the largest diameter, of the three channels. The aeration channel has the smallest clear cross-sectional area, in particular the smallest diameter. The drainage channel has, in particular, a clear cross-sectional area, in particular a diameter, which is between the clear cross-sectional area (or diameter) of the ventilation channel and of the aeration channel.

The cross-section of the tube shaft may have a symmetrical shape at least in regions of its longitudinal extent with respect to a line running in the direction of extent of the broad sides of the tube shaft, in particular the line on which the openings of the channels, in particular the areal center points thereof, are located.

The cross-section of the tube shaft may have an asymmetrical shape with respect to a line running in the direction of extent of the narrow sides of the tube shaft. In particular, the tube shaft is thicker on the side of the drainage channel in the direction transverse to the extent of the broad sides.

Roughly speaking, the cross-section can be symmetrical to the “plane” in which the channels lie and asymmetrical to a plane that extends transversely to the previous plane.

The cross-section of the tube shaft may be designed to be wider on the drainage channel side than on the aeration channel side with respect to the areal center point of the ventilation channel. To put it simply, the tube shaft is thicker, when viewed from the ventilation channel, towards the drainage channel than towards the aeration channel.

The shape of the tube shaft, due to the asymmetry described above and the curvature explained further below, promotes easy and low-trauma insertion. The shape is adapted to the slightly lateral placement of the entrance to the food pipe (esophagus) in the human body.

The curvature of the tube body mentioned above is visible when looking at the narrow sides of the cross-section of the tube shaft. When looking at the broad sides of the tube shaft, the laryngeal tube appears to be straight. The curvature, together with the flat shape and the asymmetry, facilitates the insertion of the laryngeal tube. Furthermore, the laryngeal tube can positively maintain its position and orientation when inserted, which increases the reliability of ventilation.

The ventilation opening is located, in particular, on a broad side of the tube shaft. If the tube body is curved, the ventilation opening is formed on the broad side of the tube shaft which faces the center or centers of curvature. The ventilation opening is therefore located on the inside of the curve (the curved arch shape).

In particular, the ventilation opening may extend substantially across the width of the ventilation channel. The width of the ventilation channel is the extent thereof transverse to the central longitudinal line and when looking at a broad side of the tube shaft. In other words, the ventilation opening may be as wide as the ventilation channel at its widest extent transverse to the central longitudinal line.

The tube shaft, in which the drainage channel, the ventilation channel, and the aeration channel run next to one another, in particular parallel to one another, is in particular designed asymmetrically when viewed along the central longitudinal line.

According to a second aspect, a laryngeal tube having the common features is characterized by a tube body which is designed as an injection-molded part in one piece and free of adhesive joints. In particular, the tube body may be free of joints. These characterizing features and the following features also constitute an advantageous further development of the laryngeal tube according to the first aspect.

In this variant, the drainage channel, the ventilation channel, and the aeration channel are formed as lumens in the material of the tube body. They therefore extend in the solid material of the tube body and are delimited thereby. At their proximal ends, the channels may each have inserts, connectors, connecting pieces, or the like, which are made of a different material from the tube body and may, for example, extend into the channel in the region of the proximal end thereof. The ventilation channel ends with the ventilation opening. The aeration channel ends after a connection to the esophageal cuff via which the cuff can be aerated. The drainage channel typically extends to the distal end and opens out therein. The further elements mentioned, which are arranged at the proximal end of the channels, can be held there in a force-locking manner—for example, by being inserted into the channel in a press fit. However, the further elements may also be connected to the material of the tube body in a form-fitting manner; for example, the tube body may be injection-molded onto the same. An integral bond is also possible, e.g., via an adhesive connection (the tube body itself is free of adhesive joints, in particular free of joints, which excludes the possibility that it is glued together from multiple parts, but does not exclude the possibility that something that is not part of the tube body is glued thereto).

“In one piece” and “free of adhesive joints” should, in particular, be understood to mean a design in which the entirety of the material that forms (or defines) the drainage channel, ventilation channel, and aeration channel is a continuous, in particular homogeneous, material. The laryngeal tube may comprise components that are attached to the tube body—for example, via an adhesive connection. Typically, such further components are the esophageal cuff and the pharyngeal cuff. These are typically made of a thin-walled plastic material which is firmly attached to the tube body formed in one piece and free of adhesive joints in an integrally bonded manner, in particular by means of an adhesive connection. The tube body thus forms the drainage channel, ventilation channel, and aeration channel and in this variant is made of a solid material. Further components of the laryngeal tube, in particular the esophageal cuff and the pharyngeal cuff, can be attached, in particular glued, to the solid material of the tube body.

According to a third aspect, a laryngeal tube having the common features mentioned at the outset is characterized in that the pharyngeal cuff is glued to the tube body, and the tube body has a collar which can be gripped from behind in the region of the adhesive connection to the pharyngeal cuff and into which the esophageal cuff is glued. The collar which can be gripped from behind can also be defined as a hook-like, rear-gripping portion. These characteristic features and the following features also constitute an advantageous further development of the laryngeal tube according to the previous aspects.

The collar which can be gripped from behind allows for efficient gluing over a small area. In particular, the pharyngeal cuff, or rather the material thereof, can be adhesively attached to the material of the tube body on both sides. Put simply, the pharyngeal cuff can be glued by part of its inner surface to the main body of the tube body, and by part of its outer surface to the collar that can be gripped from behind and that extends from the main body of the tube body. A slot can be formed, between the collar and the solid material of the tube body, into which the pharyngeal cuff is glued. The collar that can be gripped from behind offers further advantages with regard to the positioning of the cuff during gluing. The cuff can be positioned more easily and accurately. The cuff can be positioned and glued close to the ventilation opening using the collar. Secondary machining is usually not necessary. The collar thus simplifies manufacture and has a positive effect on the finished laryngeal tube.

The surface of the tube body may be set back in the region of the adhesive connection to the pharyngeal cuff and/or esophageal cuff by the wall thickness of the relevant cuff, such that the surface of the laryngeal tube is substantially step-free at the edge of the adhesive joint of the relevant cuff.

The aeration channel may have an aeration opening in the region of each of the two cuffs, in particular extending transversely to the central longitudinal line of the laryngeal tube, via which opening the cuffs can be inflated and/or deflated. The aeration openings may extend, in particular, in the direction of extent of the broad sides, which facilitates the manufacture of the tube body by injection molding. The aeration channel is closed at the distal end of the laryngeal tube, in particular by the solid material of the tube body. The aeration openings can be formed, in particular, by stops of a core of an injection-molding tool used to manufacture the tube body. The aeration openings are thus formed during the injection-molding step of manufacture and do not have to be subsequently incorporated into the material.

According to a fourth aspect, a laryngeal tube having the common features mentioned at the outset is characterized in that the esophageal cuff has a shape that tapers towards the drainage opening and that tapers more sharply than the shape of the esophageal cuff on the side facing away from the drainage opening. Furthermore, the esophageal cuff in this embodiment has a width extent, transverse to the tube longitudinal extent, that is greater than a depth extent of the esophageal cuff transverse to the tube longitudinal extent and to the width extent of the esophageal cuff. These characteristic features and the following features also constitute an advantageous further development of the laryngeal tube according to the previous aspects.

The esophageal cuff therefore has a shape that extends flat in the same plane as that in which the tube body itself is designed to be flat. In addition, the esophageal cuff tapers towards the distal end in a more or less arrow-shaped manner. The esophageal cuff thus tapers towards the distal end. The arrow-shaped tapering is present both when looking at the broad side of the tube body and when looking at the narrow side. The esophageal cuff can close or block the entrance to the esophagus particularly reliably, because it can adapt particularly well to the patient's anatomy. The advantageous fit of the esophageal cuff also leads to less wrinkling, since the cuff has a basic shape that corresponds to the anatomically required shape. A cuff shape that deviates from the anatomically required shape will result in wrinkling during use, which can negatively affect the seal and can also lead to irritation. The described shape of the cuff is also size-optimized. Cuffs designed differently are often larger and, when evacuated or uninflated, are flaccid and have increased folds when inserted. The folds and the flaccid cuff lead to increased irritation during insertion and pose a potential risk of injury. This is reduced or avoided by the described shape.

According to a fifth aspect, a laryngeal tube having the common features mentioned at the outset is characterized in that the pharyngeal cuff is designed such that it follows the course of the proximal edge of the ventilation opening and extends along the ventilation opening into a region of the ventilation opening in which the ventilation opening tapers in the lateral direction. The pharyngeal cuff extends along the tube longitudinal extent in the distal direction to a region in which the ventilation opening tapers in terms of its clear width or decreases in width. These characteristic features and the following features also constitute an advantageous further development of the laryngeal tube according to the previous aspects.

The pharyngeal cuff can thus efficiently and safely separate the ventilation opening from the surrounding tissue. With this design, the pharyngeal cuff can also close or block the pharynx particularly reliably and safely. At the same time, it is ensured that the ventilation opening is not accidentally blocked. The ventilation opening may, in particular, have a substantially semi-circular or circular, segment-shaped proximal edge and a distal edge that tapers to a point longitudinally towards the distal end. In the region of the distal half of the ventilation opening, said ventilation opening may transition into a distal, groove-like extension. The groove-like extension further reduces the risk of blockage and can also serve as a guide when inserting equipment or as an orientation aid when inserting an endoscope.

According to a sixth aspect, a laryngeal tube having the common features mentioned at the outset is characterized in that the ventilation opening has an, in particular rounded, proximal edge region, a central portion, which extends in a tube longitudinal direction, of the edge, and a distal edge region having edges that converge towards one another. The ventilation opening further has a support structure, in particular having two support webs, which extends from the edge of the ventilation opening into the same. The support structure serves to prevent the patient's epiglottis from folding into the ventilation opening by supporting the epiglottis if necessary, wherein the support structure extends from the proximal edge region into the central portion in the tube longitudinal direction. These characteristic features and the following features also constitute an advantageous further development of the laryngeal tube according to the previous aspects.

The tube body may be reinforced in the proximal region, which lies in the patient's bite region when the tube is inserted, in such a way that it counteracts a closure of the aeration channel due to a bite by the patient, in particular prevents a closure of the aeration channel.

### BRIEF DESCRIPTION OF DRAWINGS

Further features, details, and advantages of the invention emerge from the wording of the claims and from the following description of exemplary embodiments with reference to the drawings, in which:

FIG. 1 is a view of a laryngeal tube according to the invention;

FIG. 2 is another view of the laryngeal tube from FIG. 1;

FIG. 3 is a view of a tube body of the laryngeal tube from FIG. 1;

FIG. 4 shows a subregion of the tube body from FIG. 4 in perspectival view;

FIG. 5 shows a distal end of the tube body from FIG. 4 in perspectival view;

FIG. 6 is a sectional view along the line AA from FIG. 5;

FIG. 7 is a view of the proximal end of the tube body from FIG. 3;

FIG. 8 shows the region of application of the laryngeal tube; and

FIG. 9 shows a subregion of an alternative laryngeal tube.

### DETAILED DESCRIPTION

In the following description and in the figures, corresponding components and elements bear the same reference signs. For improved clarity, not all reference signs are reproduced in all figures.

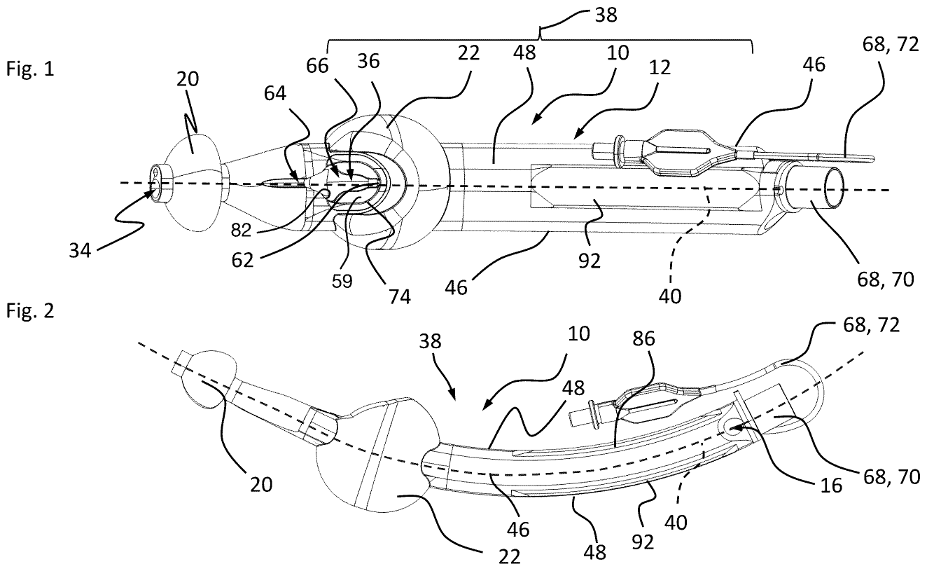

FIGS. 1 and 2 show a laryngeal tube 10 according to the invention in a top view and a side view.

The laryngeal tube 10 is designed having a tube body 12 which comprises a ventilation channel 14, a drainage channel 16, and an aeration channel 18.

On the tube body 12, an esophageal cuff 20 is arranged for blocking the esophagus 24, and a pharyngeal cuff 22 is arranged on the tube body 12 and serves to block the pharynx 26. The esophageal cuff 20 and the pharyngeal cuff 22 can be inflated and deflated via the aeration channel 18. When inserted into the patient's throat, the cuffs 20, 22 are uninflated, and are inflated when in place in order to block the esophagus 24 and pharynx 26 and to correctly position the laryngeal tube 10.

The anatomical region of application of the laryngeal tube 10 is illustrated in FIG. 8. FIG. 8 shows the throat region of a patient in a sectional view, and the esophagus 24, epiglottis 33, trachea 28, and pharynx 26 are each designated by reference signs. The pharynx 26 extends from the hyperpharynx 30 to the nasopharynx 32. The patient must be supplied with breathing air via the trachea 28. The placed laryngeal tube 10 is also shown in FIG. 8. The esophageal cuff 20 closes the esophagus 24, and the pharyngeal cuff 22 closes the pharynx 26.

The drainage channel 16 has a drainage opening 34, located distally from the esophageal cuff 20 that blocks and seals the patient's esophagus 24. In the placed state, the drainage opening 34 is located in the patient's esophagus 24 in order to drain any escaping gastric contents.

The ventilation channel 14 has a ventilation opening 36 which is arranged between the pharyngeal cuff 22 and the esophageal cuff 20. In the placed state, the ventilation opening 36 is located approximately at the level of the epiglottis 33 and at the entrance of the trachea 28. Since the esophageal cuff 20 seals the esophagus 24 in the distal direction from the ventilation opening 36, and the pharyngeal cuff 22 seals the pharynx 26 in the proximal direction, air supplied via the ventilation channel 14 is introduced into the trachea 28. The patient can in this way be ventilated.

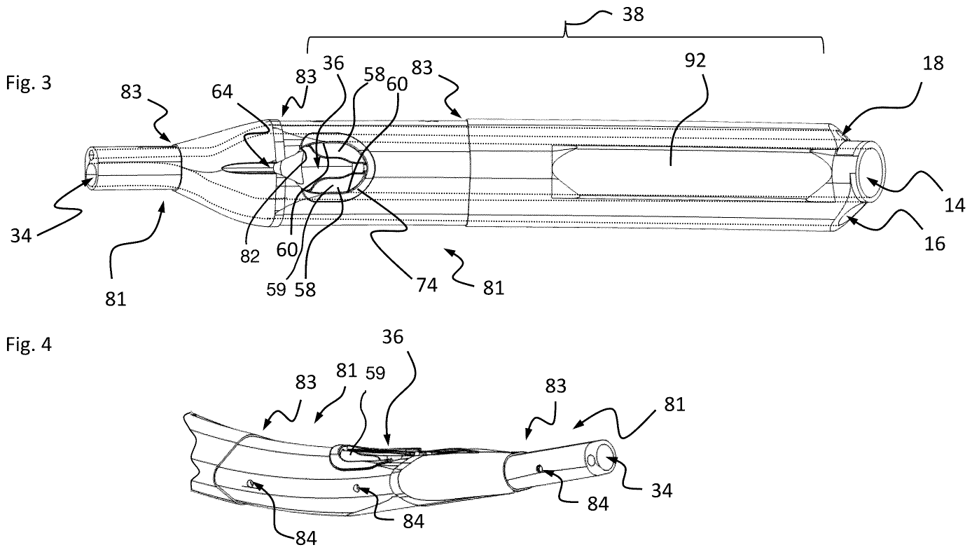

The laryngeal tube 10 according to the invention has a shape that facilitates easy insertion and minimizes the risk of injury. The tube body 12 is elongated and, overall, extends along a central longitudinal line 40. The tube body 12 comprises a tube shaft 38 in which the ventilation channel 14, the drainage channel 16, and the aeration channel 18 run parallel to one another. The course of the ventilation channel 14, drainage channel 16, and aeration channel 18 is shown in FIG. 3 by dotted lines. In FIGS. 1 and 3, the longitudinal extent of the tube shaft 38 is marked by means of curly brackets. The ventilation channel 14 does not extend further distally from the ventilation opening 36, and the drainage channel 16 and the aeration channel 18 converge towards one another.

Typically, the tube body 12, or the central longitudinal line 40 thereof, is curved at least in portions. The curvature of the tube body 12 is, in particular, designed such that the center or centers of curvature are located on one side of the tube body 12. The tube body 12 is thus curved only in one direction. The centers of curvature are located on the side of the ventilation opening 36.

The tube body 12 is flexible or is formed from a resilient material or plastic material. The tube body 12 can therefore deform when inserted into the patient's pharynx 26, allowing it to adapt to anatomical conditions and minimizing the risk of injury.

The laryngeal tube 10 is easy to insert due to its shape and material properties, and minimizes the risk of injury when inserted into the patient's pharynx 26. The laryngeal tube 10 can also be manufactured efficiently. The entire tube body 12, as shown in FIG. 3, is designed as a one-piece, injection-molded part that is free of adhesive joints. Further elements 68 can be connected to the tube body 12, which will be discussed in more detail below. The drainage channel 16, the ventilation channel 14, and the aeration channel 18 are formed as lumens in the solid material of the tube body 12.

In particular, the advantageous shape of the laryngeal tube 10 or tube body 12 according to the invention is explained in more detail below. The individual aspects of the shape of the laryngeal tube 10 or the tube body 12 thereof are also to be understood as independent inventions and are inventive not only in their entirety.

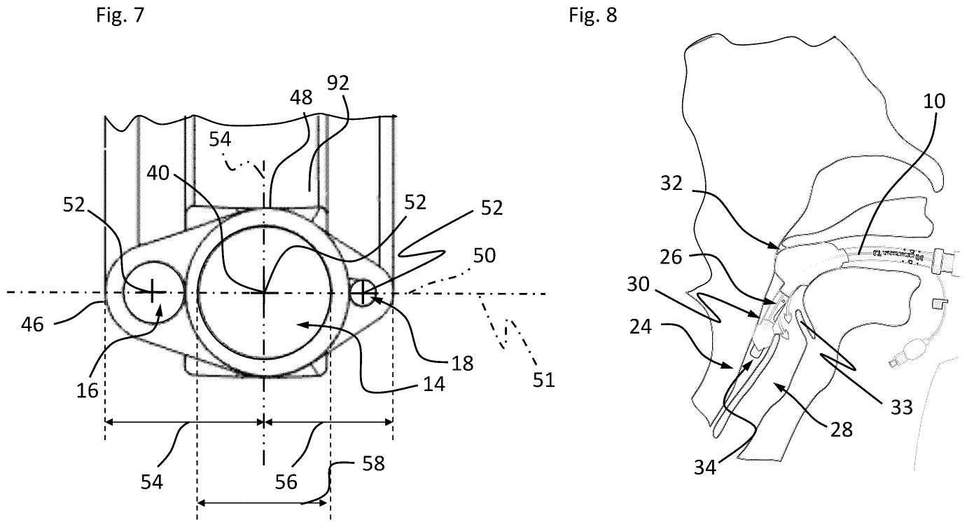

In the tube shaft 38, the ventilation channel 14 is arranged centrally and the drainage channel 16 is arranged on a first side 42 of the ventilation channel 14, and the aeration channel 18 is arranged on a second side 44 of the ventilation channel 14 that is arranged opposite the first side 42. The tube shaft 38 therefore has a flattened, cross-sectional shape, which is clearly shown, in particular, in the sectional view of FIG. 7.

The flattened, cross-sectional shape results in a tube shaft 38 which has narrow sides 46 and broad sides 48.

When looking along the central longitudinal line 40 onto a cross-sectional area of the tube shaft 38, wherein the cross-sectional area runs orthogonally to the central longitudinal line 40, the openings of the drainage channel 16, ventilation channel 14, and aeration channel 18 are located, in particular, on a straight line 50, or a straight line 50 can be drawn through the openings of all three channels 14, 16, 18 in the cross-sectional area (see, in particular, FIG. 7).

In particular, the areal center points 52 of the openings of all three channels 14, 16, 18 lie on the line 50 in the cross-sectional area. The three channels 14, 16, 18 in particular have a circular cross-section.

The ventilation channel 14 has, in particular, the largest clear cross-section, in particular diameter (in the case of a circular cross-section), of the three channels 14, 16, 18. The aeration channel 18, in particular, has the smallest clear cross-section, in particular diameter. The drainage channel 16 has, in particular, a clear cross-section, in particular a diameter, which lies between that of the ventilation channel 14 and aeration channel 18.

The cross-section of the tube shaft 38 may (when looking along the central longitudinal line 40) have a symmetrical shape at least in regions of its longitudinal extent with respect to a line 51 which runs in the direction of extent of the broad sides 48 of the tube shaft 38, in particular the line 50 on which the openings of the three channels 14, 16, 18, in particular the areal center points 52 thereof, lie.

Orthogonal to this, the cross-section of the tube shaft 38 may, in particular, have an asymmetrical shape. Accordingly, the cross-section of the tube shaft 38 may have an asymmetrical shape with respect to a line 54 that runs in the direction of extent of the narrow sides 46 of the tube shaft 38. In particular, the tube shaft 38 is thicker on the side of the drainage channel 16 in the direction transverse to the extent of the broad sides 48 than on the side of the aeration channel 18.

The cross-section of the tube shaft 38 may therefore have, with respect to the areal center point 52 of the ventilation channel 14 on the side of the drainage channel 16, a lateral extent 54 (transverse to the narrow sides 46 and parallel to the broad sides 48) that is greater than a lateral extent 56 (transverse to the narrow sides 46 and parallel to the broad sides) on the side of the aeration channel 18 (see FIG. 7). The drainage channel 16 is located, in particular, on the right side of the ventilation channel 14 when viewed from the inner side of the curvature of the tube shaft 38 (from the side of the center of curvature).

Due to the described asymmetry and curvature, the tube shaft 38 has a shape that facilitates easy and low-trauma insertion. The shape is adapted to the slightly lateral placement of the entrance of the food pipe (esophagus 24) in the human body.

The aforementioned curvature of the tube body 12 is designed in such a way that it becomes apparent when looking at the narrow sides 46 of the cross-section of the tube shaft 38. When looking at the broad sides 48 of the tube shaft 38, the laryngeal tube 10 appears to be straight (see FIGS. 1 and 2 in comparison).

The ventilation opening 36 is arranged on a broad side of the tube shaft 38. More precisely, the ventilation opening 36 is formed on the broad side of the tube shaft 38 which faces the center or centers of curvature. In the intended inserted state, the ventilation opening 36 is thus facing the entrance of the trachea 28.

The ventilation opening 36 may, in particular, extend substantially over the width of the ventilation channel 14. The width of the ventilation channel 14 is the extent thereof transverse to the central longitudinal line 40 and when looking at a broad side 48 of the tube shaft 38. In other words, the ventilation opening 36 at its greatest extent transverse to the central longitudinal line 40 may be as wide as the ventilation channel 14. A ventilation opening 36 that is as wide as possible counteracts closure of the ventilation opening 36.

On the ventilation opening 36, thin-walled support projections 58 may be arranged, which extend inwards from the lateral edge 60 of the ventilation opening 36 and form a support structure 59. The support projections 58 begin at the proximal edge 62 of the ventilation opening 36 and are spaced apart from one another there by means of a central gap. From the proximal edge 62, the support projections 58 extend along the lateral edges 60 of the ventilation opening 36 into the distal half of the ventilation opening 36. In the region of the distal half of the ventilation opening 36, the same transitions into a distal, groove-like extension 64. Although the support projections 58 reduce the clear passage area of the ventilation opening 36, they serve to support the epiglottis in case the same is bent—for example, when the laryngeal tube 10 is inserted in the direction of the trachea 28. Supporting the epiglottis prevents closure of the ventilation channel 14 by the epiglottis. The support projections 58 are set back, in particular, relative to the edge 60 of the ventilation opening 36 in the direction of the interior of the ventilation channel 14. Even when the epiglottis is supported, this still creates air outlet paths from the ventilation opening 36. The groove-like extension 64 further reduces the risk of blockage of the ventilation opening 36, because it ensures that ventilation air escapes even when the ventilation opening 36 is covered. In addition, the ventilation channel 14 has on the inside a ramp structure 66, which extends from the channel base to the groove-like extension 64, such that, for example, an endoscope or another device that is introduced through the ventilation channel 14 is guided through the ramp structure 66 out of the ventilation channel 14 and through the ventilation opening 36. The groove-like extension 64 can serve as a guide or as an orientation aid.

As already mentioned, the tube body 12 is designed as a one-piece, injection-molded part that is free of adhesive joints. In particular, the tube body 12 may be designed without joints, i.e., not only may it have no adhesive joints, but it may have no joints at all. The drainage channel 16, the ventilation channel 14, and the aeration channel 18 are formed as lumens in the material of the tube body 12. They therefore extend in the solid material of the tube body 12 and are delimited thereby (by the solid material). This is in contrast to tube bodies of the prior art, in which individual channels are adhesively connected to one another.

In the present example, further elements 68 are attached to the tube body 12. For connection to further devices, e.g., a cannula for aerating the cuffs or a ventilation bag for aerating the ventilation channel 14, the laryngeal tube 10 may have inserts, connectors, connecting pieces, or the like at its proximal end which are made of a different material from the tube body 12 and may extend, for example, into the relevant channel 14, 16, 18 in the region of its proximal end. The further elements 68 of this kind, which are arranged at the proximal end of the channels 14, 16, 18, can be held there in a force-fitting manner—for example, by being inserted into the channel 14, 16, 18 in a press fit. However, the further elements 68 may also be connected in a form-fitting manner to the material of the tube body 12; for example, the tube body 12 may be injection-molded onto said further elements. In the present example, the ventilation channel 14 has a connection adapter 70 for a ventilation bag (not shown), and in the aeration channel 18, there is a connection line 72 via which air can be supplied to the aeration channel 18 in order to supply the cuffs 20, 22 with air.

A one-piece tube body 12 that is free of adhesive joints is thus designed, in particular, such that the entirety of the material which forms (or defines) the drainage channel 16, ventilation channel 14, and aeration channel 18 is a continuous, in particular homogeneous, material. The laryngeal tube 10 may in turn comprise components that are attached to the tube body 12—for example, via an adhesive connection.

Typically, such further components are the esophageal cuff 20 and the pharyngeal cuff 22 and, if applicable, the further elements 68 mentioned above.

The cuffs, i.e., the esophageal cuff 20 and the pharyngeal cuff 22, are typically made of a thin-walled plastic material that is firmly attached to the one-piece tube body 12, which is free of adhesive joints, in an integrally bonded manner, in particular by means of an adhesive connection.

In the example shown in the figures, the esophageal cuff 20 and/or the pharyngeal cuff 22 are glued to the tube body 12. The tube body 12 has, in the region of the adhesive connection to the pharyngeal cuff 22 around the ventilation opening 36, a hook-like, rear-gripping portion 74 or collar 74 that can be gripped from behind.

The tube body 12 thus forms a rear-gripping portion or collar that can be gripped from behind, which portion or collar is surrounded by the material of the pharyngeal cuff 22. The pharyngeal cuff 22 is glued into this collar 74 that can be gripped from behind. The effective adhesive area is thereby provided on the top and bottom of the material of the pharyngeal cuff 22, such that an adhesive connection sufficient for attachment can be provided on a smaller overall area. FIG. 5 shows a subregion of the tube body 12 in detail, and FIG. 6 shows a section along the line A-A in FIG. 5. In the sectional view, it is clearly visible that the rear-gripping portion 74 or the collar 74 that can be gripped from behind forms a slot 76 into which the pharyngeal cuff 22 can be inserted such that it can be adhesively attached both to the bottom 78 of the slot 76 and to the top 80 of the slot 76.

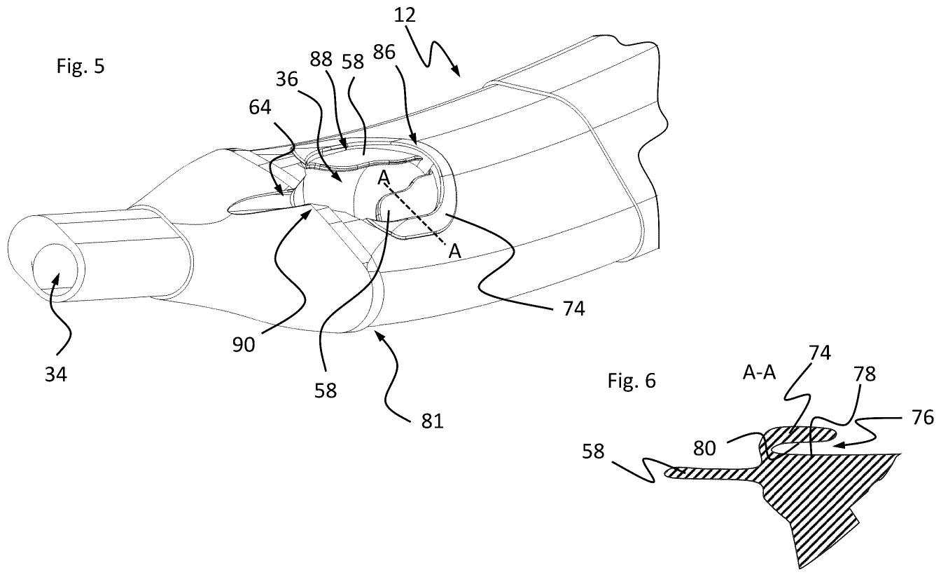

As can be clearly seen in FIGS. 3 to 5, in which the tube body 12 is shown without the cuffs 20, 22, the surface of the tube body 12 in the region of the adhesive connection to the pharyngeal cuff 22 and esophageal cuff 20 is set back by the wall thickness of the relevant cuff 20, 22, or a region 81 to be glued is set back. As a result, step-like transitions 83 are formed on the tube body 12 in each case (FIGS. 3 and 4). In this way, the surface of the laryngeal tube at the edge of the adhesive joint of the relevant cuff 20, 22 can be designed to be substantially step-free when the cuff 20, 22 is glued on, which can be seen in FIGS. 1 and 2.

The esophageal cuff 20 has a shape that tapers towards the drainage opening 34 and that tapers more sharply than the shape of the esophageal cuff 20 on the side facing away from the drainage opening 34. The esophageal cuff 20 is therefore tapered “to a point” distally and flat proximally. The esophageal cuff 20 further has a width extent (viewing direction from FIG. 1), transverse to the tube longitudinal extent (along the central longitudinal line 40) and when looking at the broad side 48, which is greater than a depth extent (viewing direction from FIG. 2) of the esophageal cuff 20 transverse to the tube longitudinal extent and when looking at the narrow side 46.

The esophageal cuff 20 thus has a shape that extends flat in the plane in which the tube body 12 itself is also designed to be flat. In addition, the esophageal cuff 20 tapers towards the distal end in an arrow-shaped manner. The esophageal cuff 20 thus tapers towards the distal end. The arrow-shaped convergence can be seen when looking at the broad side 48 of the tube body 12 and also when looking at the narrow side 46. The esophageal cuff 20 can close or block the entrance to the esophagus particularly reliably, because it can adapt particularly well to the patient's anatomy.

The pharyngeal cuff 22 is designed such that it follows the course of the proximal edge 62 of the ventilation opening 36 and extends along the ventilation opening 36 into a region of the ventilation opening 36 that tapers in the distal direction and that transitions into the distal, groove-like extension 64. In this design, the pharyngeal cuff 22 can close or block the pharynx 26 particularly reliably and safely. At the same time, it is ensured that the ventilation opening 36 is not accidentally blocked, since the pharyngeal cuff 22 extends distally around the ventilation opening 36. The proximal edge 62 of the ventilation opening 36 is, in the present case, substantially semi-circular or circular segment-shaped. A distal edge 82, however, is tapered distally in the longitudinal direction and transitions into the distal, groove-like extension 64 and tapers to a point.

The cuffs 20, 22 are inflated and deflated via lateral outlets 84 on the ventilation channel 18, which can be seen in FIG. 4.

The ventilation opening 36 or the edge region thereof has an, in particular rounded, proximal edge 62, a central portion 88 that extends in a tube longitudinal direction, and a distal edge region 82 having edges converging towards one another. The support structure 59 of the ventilation opening 36, which, in the present example, is formed by the two support webs 58, extends from the proximal edge region 62 and from the central portion 88 inwards into the ventilation opening 36.

The tube body 12 is designed having reinforcing structures 92 in the proximal region, which lies in the patient's bite region when the tube is inserted. The reinforcing structures 92 counteract a closure of the aeration channel 18 due to a bite by the patient.

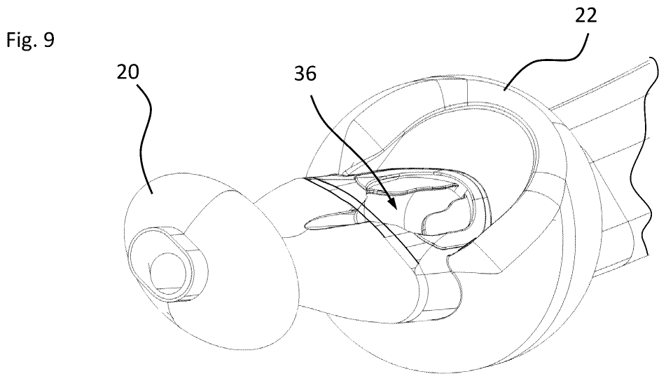

FIG. 9 shows the distal end of an alternative laryngeal tube 10, having a tube body 12 which is designed as a one-piece, injection-molded part that is free of adhesive joints, like the laryngeal tube 10 illustrated in FIGS. 1 to 7, but has a simplified region around the ventilation opening 36, without the support structure 59 and without the collar 74 that can be gripped from behind. However, the cuffs 20 and 22 are glued to the tube body 12, as in the example of FIGS. 1 to 7.

1. A laryngeal tube for ventilating a patient, the laryngeal tube comprising: an elongated tube body which is designed having a ventilation channel, a drainage channel, and an aeration channel, wherein an esophageal cuff for blocking an esophagus and a pharyngeal cuff for blocking a pharynx are arranged on the tube body, wherein the drainage channel has a drainage opening which is arranged distally from the esophageal cuff, and the ventilation channel has a ventilation opening which is arranged between the pharyngeal cuff and the esophageal cuff, wherein the esophageal cuff and/or the pharyngeal cuff is/are inflatable and/or deflatable via the aeration channel, and wherein the tube body has a tube shaft in which the ventilation channel is arranged centrally and the drainage channel is arranged on a first side of the ventilation channel, and the aeration channel is arranged on a second side of the ventilation channel that is opposite the first side, such that the tube shaft has a flattened cross-sectional shape.

2. The laryngeal tube for ventilating a patient, the laryngeal tube comprising: an elongated tube body which is designed having a ventilation channel, a drainage channel, and an aeration channel, wherein an esophageal cuff for blocking an esophagus and a pharyngeal cuff for blocking a pharynx are arranged on the tube body, wherein the drainage channel has a drainage opening which is arranged distally from the esophageal cuff, and the ventilation channel has a ventilation opening which is arranged between the pharyngeal cuff and the esophageal cuff, wherein the esophageal cuff and/or the pharyngeal cuff is/are inflatable and/or deflatable via the aeration channel, wherein the tube body is designed in one piece and free of adhesive joints, or free of joints, and as an injection-molded part, wherein the esophageal cuff and the pharyngeal cuff are adhesively attached to the tube body.

3. The laryngeal tube for ventilating a patient, the laryngeal tube comprising: an elongated tube body which is designed having a ventilation channel, a drainage channel, and an aeration channel, wherein an esophageal cuff for blocking an esophagus and a pharyngeal cuff for blocking a pharynx are arranged on the tube body, wherein the drainage channel has a drainage opening which is arranged distally from the esophageal cuff, and the ventilation channel has a ventilation opening which is arranged between the pharyngeal cuff and the esophageal cuff, wherein the esophageal cuff and/or the pharyngeal cuff is/are inflatable and/or deflatable via the aeration channel, wherein the pharyngeal cuff is glued to the tube body and the tube body has a collar which can be gripped from behind in the region of the adhesive connection to the pharyngeal cuff and into which the pharyngeal cuff is glued, wherein the collar which can be gripped from behind forms a slot in which the material of the pharyngeal cuff is received and is contacted on two sides by the material of the tube body.

4. The laryngeal tube for ventilating a patient, the laryngeal tube comprising: an elongated tube body which is designed having a ventilation channel, a drainage channel, and an aeration channel, wherein an esophageal cuff for blocking an esophagus and a pharyngeal cuff for blocking a pharynx are arranged on the tube body, wherein the drainage channel has a drainage opening which is arranged distally from the esophageal cuff, and the ventilation channel has a ventilation opening which is arranged between the pharyngeal cuff and the esophageal cuff, wherein the esophageal cuff and/or the pharyngeal cuff is/are inflatable and/or deflatable via the aeration channel, wherein the esophageal cuff has a shape that tapers towards the drainage opening and that tapers more sharply than the shape of the esophageal cuff on a side facing away from the drainage opening, and the esophageal cuff has a width extent, transverse to the tube longitudinal extent, that is greater than a depth extent of the esophageal cuff that runs transverse to the tube longitudinal extent and to the width extent of the esophageal cuff.

5. The laryngeal tube for ventilating a patient, the laryngeal tube comprising: an elongated tube body which is designed having a ventilation channel, a drainage channel, and an aeration channel, wherein an esophageal cuff for blocking an esophagus and a pharyngeal cuff for blocking a pharynx are arranged on the tube body, wherein the drainage channel has a drainage opening which is arranged distally from the esophageal cuff, and the ventilation channel has a ventilation opening which is arranged between the pharyngeal cuff and the esophageal cuff, wherein the esophageal cuff and/or the pharyngeal cuff is/are inflatable and/or deflatable via the aeration channel, wherein the pharyngeal cuff is designed such that the pharyngeal cuff follows the course of a proximal edge of the ventilation opening and extends along an edge of the ventilation opening in a distal direction into a region of the ventilation opening in which the ventilation opening tapers in a lateral direction.

6. The laryngeal tube for ventilating a patient, the laryngeal tube comprising: an elongated tube body which is designed having a ventilation channel, a drainage channel, and an aeration channel, wherein an esophageal cuff for blocking an esophagus and a pharyngeal cuff for blocking a pharynx are arranged on the tube body, wherein the drainage channel has a drainage opening which is arranged distally from the esophageal cuff, and the ventilation channel has a ventilation opening which is arranged between the pharyngeal cuff and the esophageal cuff, wherein the esophageal cuff and/or the pharyngeal cuff is/are inflatable and/or deflatable via the aeration channel, wherein the ventilation opening has an rounded proximal edge region, a central portion, which extends in a tube longitudinal direction, of the edge, and a distal edge region having edges that converge towards one another, wherein the ventilation opening further has a support structure, or two support projections, which extends from the edge of the ventilation opening into the same in order to prevent the patient's epiglotis from folding into the ventilation opening by supporting the epiglotis, wherein the support structure extends from the proximal edge region at least into the central portion in the tube longitudinal direction.

7. The laryngeal tube for ventilating the patient, according to claim 1, wherein the tube body in the proximal region, which lies in the patient's bite region when the laryngeal tube is inserted, is reinforced in such a way that the tube body counteracts a closure of the aeration channel due to a bite by the patient, to prevent a closure of the aeration channel.

8. The laryngeal tube for ventilating the patient, according to claim 1, wherein the surface of the tube body in the region of the adhesive connection to the pharyngeal cuff is set back by a wall thickness of the pharyngeal cuff, such that the surface of the laryngeal tube is substantially step-free at the edge of the adhesive joint of the pharyngeal cuff.
